(19) United States
(12) Patent Application Publication
(10) Pub. No.: US 2015/0174387 A1
(43) Pub. Date: Jun. 25, 2015

(54) COMBINED SONIC AND ULTRASONIC SKIN CARE DEVICE

(71) Applicant: L'Oreal, Paris (FR)

(72) Inventors: James Christopher McInnes, Seattle, WA (US); Ken Pilcher, Redmond, WA (US); Zane Miller, Seattle, WA (US)

(73) Assignee: L'OREAL, Paris (FR)

(21) Appl. No.: 14/139,423

(22) Filed: Dec. 23, 2013

(51) Int. Cl. A61M 37/00

(52) U.S. Cl. CPC A61M 37/0092; A61M 2210/04; A61M 2205/058; A61M 2205/106

(57) ABSTRACT

Dermal infusion devices and methods are provided. The device is configured to direct two different frequencies of oscillating motion (e.g., sonic and ultrasonic) towards the skin of a subject. The combination of the two different frequencies results in improved dermal infusion capabilities and can be used, for example, to effect transdermal delivery of a topical formulation.

### BACKGROUND

The non-invasive, transdermal delivery of both low and high molecular weight therapeutic agents has long been the goal of researchers around the world. Even ignoring the desire to eliminate invasive delivery techniques, such as hypodermic injection, that are painful and can open the skin to infection, there are many cases where the condition being treated is in the skin itself. Whether the condition is pathological or cosmetic, in most cases the standard method of care is the application of a topical agent that is intended to be absorbed directly through the horny layer of the skin and penetrate to the epidermis or dermis layers where treatment occurs. However, the normal function of skin runs precisely counter to this kind of treatment. Keratinized cells and multiple lipid bilayers in the stratum corneum present a significant barrier, not only to infectious or harmful agents, but also to great many cosmetic treatments and external drug preparations. The result is that for most topical preparations, if flux of the active agent across the stratum corneum happens at all, it happens at a very slow rate, requiring longer treatment times and higher dosages.

To overcome this barrier in a noninvasive fashion, several techniques have been developed to increase transdermal flux. Generally, they fall into two categories. The first category attempts to increase flux by increasing the permeability of the skin. This group includes adding chemicals to the topical formulation that cause the skin to allow greater penetration, encapsulating the active agent in lipophilic molecules that pass easier through the lipid bilayers, and techniques such as electroporation and sonophoresis that cause temporary, reversible micropores in the lipid bilayers. The second category attempts to increase transdermal flux by creating an energy gradient that causes the active ingredient to travel down that gradient across the stratum corneum (and deeper, if desired). This group includes iontophoresis and magnetophoresis, which use electrical and magnetic fields respectively.

Several approaches to increasing flux combine two or more of the techniques listed above. For example, U.S. Pat. No. 7,427,273, incorporated herein by reference in its entirety, describes a system that incorporates both sonophoresis and iontophoresis.

Despite progress in the field to date, improved techniques for transdermal delivery are still desired.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a dermal infusion device is provided. In one embodiment, the device includes a first source of oscillatory motion, having an oscillation rate of less than 20 kHz; and a second source of oscillatory motion, having an oscillation rate of 20 kHz or greater.

In another aspect, a method of simultaneously delivering a sonic motion and an ultrasonic motion to a location is provided. In one embodiment, the method includes the steps of:

(a) directing the sonic motion at the location from a first source of oscillatory motion, having an oscillation rate of less than 20 kHz; and

(b) directing the ultrasonic motion at the location from a second source of oscillatory motion, having an oscillation rate of 20 kHz or greater.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A schematically illustrates a device in accordance with the disclosed embodiments;

FIGS. 1B-1D schematically illustrate the mechanism by which the disclosed embodiments enhance transdermal delivery;

FIG. 2 illustrates representative absorption spectra for two modes of treatment: finger and sonic/ultrasound in accordance with the disclosed embodiments;

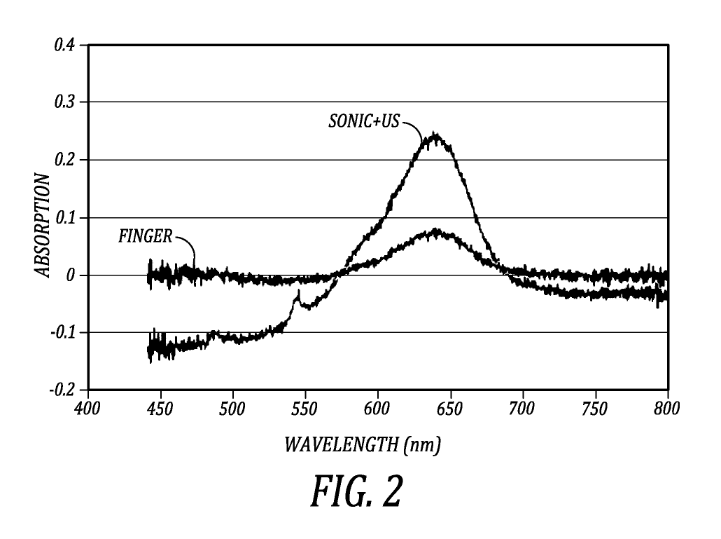

FIG. 3 is a graphical representation of skin absorption data obtained from exemplary devices in accordance with the disclosed embodiments;

FIG. 4 is a graphical representation of skin absorption data obtained from exemplary devices in accordance with the disclosed embodiments;

FIG. 5A illustrates the relationship of the ultrasound burst timing and with respect to the sonic motion of the device;

FIG. 5B relates to FIG. 5A and is a graphical representation of skin absorption data obtained from exemplary devices in accordance with the disclosed embodiments.

FIG. 6A illustrates the relationship of the ultrasound burst timing and intensity with respect to the sonic signal;

FIG. 6B relates to FIG. 6A and is a graphical representation of skin absorption data obtained from exemplary devices in accordance with the disclosed embodiments;

FIG. 7A illustrates the relationship of the ultrasound burst timing and duty cycle with respect to the sonic signal;

FIG. 7B relates to FIG. 7A and is a graphical representation of skin absorption data obtained from exemplary devices in accordance with the disclosed embodiments; and

FIG. 8 is a graphical representation of skin absorption data obtained from exemplary devices in accordance with the disclosed embodiments.

### DETAILED DESCRIPTION

Dermal infusion devices and methods are provided that improve dermal delivery of a topical formulation. In the description herein, the terms “infusion” and “absorption” are used interchangeably. The devices are configured to direct two different frequencies of oscillating motion (e.g., sonic and ultrasonic) towards the skin of a subject. The combination of the two different frequencies results in improved dermal infusion capabilities and can be used, for example, to effect dermal delivery of a topical formulation. By improving dermal delivery, the disclosed embodiments can reduce the treatment time required to deliver an effective amount of formula. Another significant advantage of this invention is that it is can be agent agnostic, meaning that because it mechanically increases the permeability of the stratum corneum, it does not rely on charge, hydrophilicity, or diamagnetic properties of the cosmetic or therapeutic agent to increase flux.

The disclosed embodiments seek to address the problem of long application times by adding an additional method of delivering disruptive energy to the skin system. In this case, the primary energy modality applied is mechanical (sonic), inducing small strain in the skin to increase permeability. The secondary energy modality applied is acoustic (ultrasonic), inducing mechanical forces and vibrations that aid in molecule transport into the epidermis. This results in a combined mechanical—acoustical device that enables multiple skin care actions. One is to massage and stimulate the skin via repeated mechanical cycles of stretching and relaxation. Another is to increase delivery and penetration of active ingredients into the epidermis.

While the various aspects of the present disclosure are presented with examples related to skin care, it will be appreciated that the disclosed examples are illustrative in nature, and therefore, should not be construed as limited to skin care applications. It should therefore be apparent that these various aspects of the present disclosure have wide application to any dermal delivery.

In one aspect, a dermal infusion device is provided. In one embodiment, the device includes a first source of oscillatory motion, having an oscillation rate of less than 20 kHz; and a second source of oscillatory motion, having an oscillation rate of 20 kHz or greater.

Similarly, in another aspect, a method of simultaneously delivering a sonic motion and an ultrasonic motion to a location is provided. In one embodiment, the method includes the steps of:

(a) directing the sonic motion at the location from a first source of oscillatory motion, having an oscillation rate of less than 20 kHz; and

(b) directing the ultrasonic motion at the location from a second source of oscillatory motion, having an oscillation rate of 20 kHz or greater.

The device provides two different sources of oscillatory motion in order to produce signals of different frequency. The first source of oscillatory motion, having an oscillation rate of less than 20 kHz, typically produces “sonic” motion. Therefore, the first source is sometimes referred to herein as a “sonic source” and its motion may be referred to as a “sonic signal.” In one embodiment, the first source has an oscillation rate of less than 1 kHz. In one embodiment, the first source has an oscillation rate of less than 200 Hz. In one embodiment, the first source has an oscillation rate of greater than 10 Hz.

In one embodiment, the first source is selected from the group consisting of a motor, a pneumatic device, and a piezoelectric device. Such sources of oscillating motion at sonic frequencies are known to those of skill in the art and can be implemented in the disclosed device accordingly.

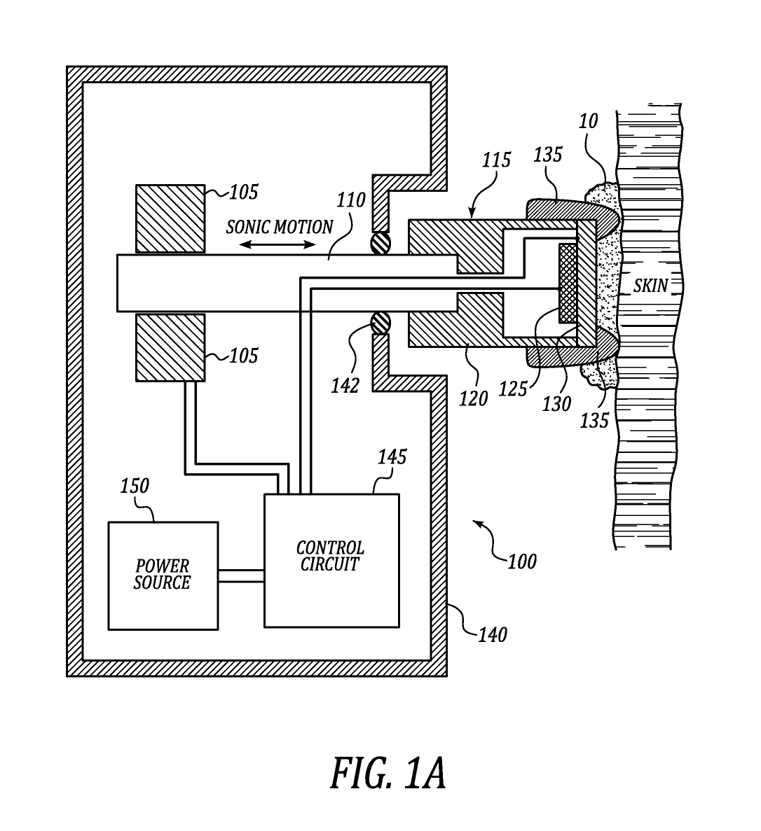

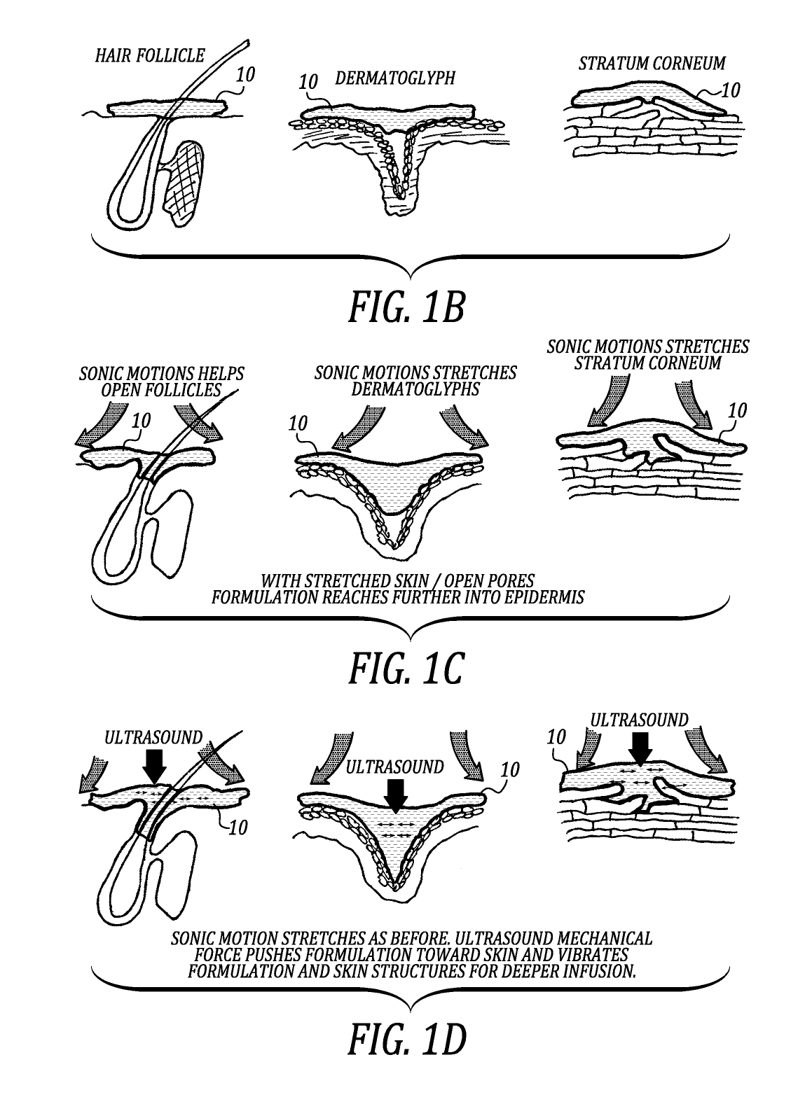

An exemplary device for providing a sonic movement is the Opal (Clarisonic, Redmond, Wash.). In this device, the applicator tip creates strain on the skin immediately adjacent to the area of the skin that is in contact with the applicator. U.S. Patent Application Publication No. 2009/0306577, incorporated herein by reference in its entirety, describes an exemplary reciprocating device (such as the Opal) that can apply a sonic motion through an applicator tip. This action increases skin permeability by temporarily flexing and enlarging dermatoglyphs, paracellular spaces or transappendageal pathways such as hair follicles and sweat glands (see FIGS. 1B-1D), which in turn increases dermal delivery. The action of the applicator tip, which is substantially perpendicular to the skin, also acts to drive a skin care product into the epidermis. This driving force occurs regardless of the formulation of the skin care product.

The second source of oscillatory motion, having an oscillation rate of 20 kHz or greater, typically refers to “ultrasonic” motion. Therefore, the second source is sometimes referred to herein as an “ultrasonic source” and its motion may be referred to as an “ultrasonic signal.” In one embodiment, the second source has an oscillation rate greater than 1 MHz. In one embodiment, the second source has an oscillation rate greater than 5 MHz. In one embodiment, the second source has an oscillation rate less than 50 MHz.

In one embodiment, the second source is selected from the group consisting of a piezoelectric device a magnetostrictive device, an electromagnetic device, a pneumatic (whistle) device and a surface wave device. Such sources of oscillating motion at ultrasonic frequencies are known to those of skill in the art and can be implemented in the disclosed device accordingly.

The sonic and ultrasonic signals each impact the skin of a subject at the same location, but in different ways, so as to facilitate transdermal delivery of a formulation or the like. In one embodiment, the sonic source and the ultrasonic source are configured to simultaneously direct oscillatory motion to the same location.

The formulation for which the device is used contains one or more ingredients desired to be driven into the epidermis of the subject. In one embodiment, the formulation is a dermatological composition. Representative dermatological compositions include (a) econazole and its salts, such as sodium, potassium, lithium, calcium, magnesium, nitrate or ammonium salts; (b) flavones, such as flavone, apigenine, chrysine, flavanone, quercetine; and (c) retinoic acid, which falls under the following categories: antifungal, antibacterial, metabolic potentiator, and vitamin.

In one embodiment, the formulation is a cosmetic composition. Representative cosmetic compositions may comprise one or more slimming agents, humectants or moisturizers, anti-ageing substances, in particular “anti-wrinkle” substances, anti-oxidants, fat-restructuring substances, substances acting on the micro-circulation, biological active substances known for their actions on the mechanotransduction chain, and tensioning agents which fix the immediate deformations conferred by the suction on the surface of the skin and thereby lead to a temporary smoothing of the skin.

In one embodiment, the formulation is applied to the applicator tip prior to contacting the subject's skin. In one embodiment, the formulation is applied to the location on the subject's skin prior to the steps of directing the sonic motion and directing the ultrasonic motion. In one embodiment, the formulation improves action between the location and the first source of oscillatory motion. In one embodiment, the formulation improves action between the location and the second source of oscillatory motion.

The formulation may enhance sonic motion by any of the following: lubricating the interface; coupling the energy transfer from the applicator tip to the skin; or having a viscosity, consistency or composition such that the formulation remains near the applicator tip during movement of the tip across the skin. The formulation may enhance ultrasonic motion by any of the following: transmitting the ultrasonic energy; providing a desirable acoustic impedance for effective energy transfer; coupling the energy transfer from the ultrasonic source to the skin; having a viscosity, consistency or composition such that the formulation effectively coats and then remains in contact with both the surface of the ultrasonic source and the surface of the skin; or discourages the formation of bubbles or foam within the formulation.

In the following description, numerous specific details are set forth in order to provide a thorough understanding of one or more embodiments of the present disclosure. It will be apparent to one skilled in the art, however, that many embodiments of the present disclosure may be practiced without some or all of the specific details. In some instances, well-known process steps have not been described in detail in order to not unnecessarily obscure various aspects of the present disclosure. Further, it will be appreciated that embodiments of the present disclosure may employ any combination of features described herein.

Turning now to FIG. 1A, there is shown one example illustrated in cross section of a dermal infusion device, generally designated 100, formed in accordance with aspects of the present disclosure. The device 100 includes a sonic source 105 (i.e., the first source of oscillatory motion) and an ultrasonic source 125 (i.e., the second source of oscillatory motion). Both the sonic source 105 and the ultrasonic source 125 are directed towards the skin of the subject at a common location. The sonic and ultrasonic motion provided by the device 100 effect transdermal delivery of a formulation 10 (e.g., a skin care formulation) into the skin.

The sonic source 105 provides sonic motion to the location via a shaft 110 operatively connected to an applicator tip 115. The applicator tip 115 terminates in a concave soft contact member 135 configured to gently contact the skin with a pocket suitable for containing the formulation 10 therebetween. Therefore, the sonic source 105 ultimately applies sonic motion to the skin via the soft contact member 135. In one embodiment, the soft contact member 135 is formed from a materials such as an elastomeric material such as silicone rubber, soft enough to avoid discomfort or injury to the skin but firm enough to maintain its shape and impart sufficient sonic energy. Other exemplary materials can also be used, such as natural rubber, butyl rubber, and polyurethane. In certain embodiments, the soft contact member 135 improves transmission of the ultrasound by containing and moving the formulation 10 so as to facilitate contact between the applicator tip 115 and skin.

In operation, the sonic source 105 reciprocates the applicator tip 115 at an amplitude of 0.075 inch to 0.1 inch.

The ultrasonic source 125 is contained within a housing 120 of the applicator tip 115. In the illustrated embodiment, the placement of the ultrasound source 125 is on the central axis of the shaft 110 and applicator tip 115. However, it will be appreciated that other arrangements are also contemplated.

The ultrasonic source 125 transmits an ultrasonic signal to the skin via an ultrasound matching layer 130. In one embodiment, the ultrasound matching layer 130 is formed from a materials such as a metal, a ceramic, a plastic, a graphite, a rubber, a glass, an epoxy, an elastomer, and a gel. The matching layer 130 acts both as a barrier to isolate the ultrasonic source 125 and as a medium through which the ultrasonic signal travels. Selection of a matching layer 130 material with the appropriate acoustic impedence helps to improve ultrasonic transmission from the ultrasonic source 125 to the location. Further, selection of the matching layer 130 material with the appropriate thickness , e.g. one half the wavelength or one quarter the wavelength of the sound within the material, helps to improve the acoustic output.

In the representative device of FIG. 1A, a housing 140 provides a grip/handle for a user of the device 100. The housing 140 contains the sonic source 105 and partially encloses the shaft 110, which passes from the housing 140, towards the applicator tip 115 via an aperture fitted with a gasket 142 configured to allow oscillating motion of the shaft 110 while sealing the inner chamber of the housing 140.

The housing 140 further contains a control circuit 145 (e.g., a printed circuit board, field-programmable gate array, ASIC, etc.) configured to operatively control the sonic source 105 and the ultrasonic source 125. As will be described in more detail below, the sonic and ultrasonic signals can be coordinated with regard to timing, intensity, and/or duration. These are controlled by the control circuit 145. A power source 150 is contained within the housing 140 and powers the control circuit 145, the sonic source 105, and the ultrasonic source 125. Power requirements will depend on the ultimate function of the device. For ultrasound, voltages in the range of 1-1000 V (peak to peak) may be required. With appropriate circuitry a battery pack can typically handle the required load, or wall power can be used.

Referring now to FIGS. 1B-1D, the mechanism by which the transdermal delivery device enhances delivery of a formulation 10 into the subject's skin is illustrated. The illustrated structures in FIG. 1B include a hair follicle, a dermatoglyph, and the stratum corneum. In FIG. 1C, sonic motion is applied to the structure in order to “open” the structure to receive a portion of the formulation 10. In FIG. 1D, ultrasonic motion is applied in order to further drive the formulation 10 into the structures. As will be discussed further below, the timing and duration of the sonic and ultrasonic signals can be coordinated based on the desired effect. Therefore, while FIGS. 1C and 1D illustrate sonic first, then ultrasonic, it will be appreciated that the signals could be applied simultaneously in reverse order.

In operation of the device, the sonic motion is typically constant over the course of a treatment time window, during which ultrasound is applied. Ultrasound may be continuous or pulsed. If pulsed, the pulse repetition frequency may be synchronized to the reciprocating motion of the device tip (e.g. emitting a pulse of ultrasound on each sonic cycle, every other cycle, every third cycle, twice per cycle, 3 times per cycle, etc.) The ultrasound frequency or intensity may be swept within a burst. Sweeping the frequency may alter peak wavefront with the ultrasound field (as taught by U.S. Pat. No. 5,444,611, incorporated herein by reference in its entirety) and align it with the reciprocating shaft delivering sonic motion. The ultrasound may have multiple frequencies, e.g. one for the transappendageal pathway and one for the paracellular pathway.

The choice of ultrasound parameters (frequency, intensity, delivery protocol) typically will focus on achieving the most desirable flux of formulation into the epidermis while minimizing any undesirable sensory stimulation for the subject. The ultrasound parameters can also be chosen to be within safe ranges published in the industry and by appropriate regulatory agencies.

Duty cycle is one ultrasound parameter that can be adjusted in order to provide a particular effect. Duty cycle is defined as the percentage of time in which the ultrasound signal is applied during one oscillation (i.e., from fully extended position back to fully extended position) of the sonic motion of the device tip. As will be discussed in the examples below, duty cycle proportionally affects the pressure and intensity applied to the skin. In one embodiment, the ultrasound source creates bursts of oscillatory motion with a duty cycle between 5% and 50%. In one embodiment, the ultrasound source creates bursts of oscillatory motion with a duty cycle between 10% and 30%. Spatial peak, temporal average intensity (I_{SPTA}) is another key characteristic of the ultrasound signal that can be controlled. I_{SPTA} is used to characterize the intensity of the ultrasound signal over a period of time. Sufficient intensity is required to effect the formulation-driving nature of the disclosed embodiments, but excessive intensity will be uncomfortable to the subject. In one embodiment, the ultrasound source has a spatial peak, temporal average intensity (I_{SPTA}) of 1 W/cm² or less. In one embodiment, the ultrasound source has a spatial peak, temporal average intensity (I_{SPTA}) of 720 mW/cm² or less. In one embodiment, the ultrasound source has a spatial peak, temporal average intensity (I_{SPTA}) of 5 mW/cm² or more.

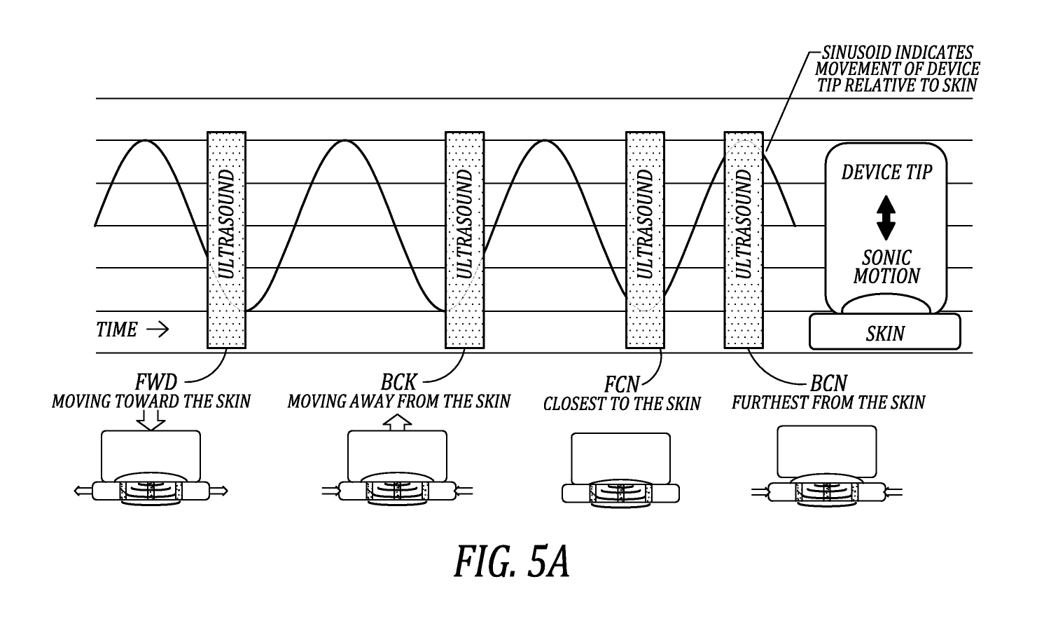

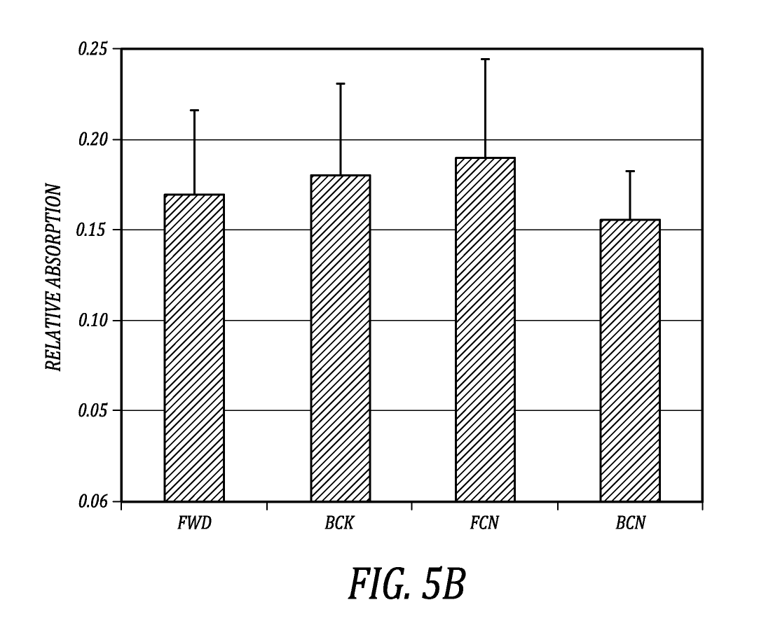

As discussed previously, in one embodiment, the sonic motion is produced by an oscillating contact member (e.g. the applicator tip 115) that oscillates between an extended position, closest to a location on the subject's skin, and a retracted position, furthest from the location. The sonic motion can be coordinated with the ultrasonic motion to provide particular effects. As described in the examples below and illustrated in FIGS. 5A and 5B, the point of the sonic oscillation cycle at which the ultrasound signal is applied will effect absorption of a formula.

In one embodiment, the ultrasonic motion impinges upon the location and is delivered such that the ultrasonic motion is centered about the instance when the contact member is in the extended position. This is the “FCN” position of FIGS. 5A and 5B and has been shown to be the most effective timing.

In one embodiment, the ultrasonic motion impinges upon the location and is delivered when the contact member is moving from the retracted position to the extended position. This is the “FWD” position of FIGS. 5A and 5B.

In one embodiment, the ultrasonic motion impinges upon the location and is delivered when the contact member is moving from the extended position to the retracted position. This is the “BCK” position of FIGS. 5A and 5B.

Exemplary Results

### 1. Methods

Experimental testing was divided into five successive parts. Parts 1 through 4 utilized blue dye as a surrogate marker for absorption and spectrophotometry to produce a relative measure of absorption. Part 5 utilized fluorescein as a surrogate marker for absorption and tape stripping to produce an actual measure of absorption. The surrogate markers were included in a formulation (Clarisonic Anti-Aging Sea Serum) specifically designed for the sonic device and also appropriate for the ultrasonic signal. Physical application of the formulation was as recommended in the sonic device instruction manual at durations of either 30 or 60 seconds. For each experiment multiple replicates (n=9-25) were run with the results being averaged per treatment condition.

i. Skin Specimen Preparation

Porcine skin (ear and flank) specimens for in-vitro testing were obtained and gently cleaned and stored for experimentation.

ii. Sonic/Ultrasonic Conditions

A modified device that applies sonic motion to the skin (Clarisonic Opal) unit was used for testing. The two primary modifications were: 1) addition of an ultrasonic module within the sonically active applicator tip with and 2) a means to output the motor drive signal so as to synchronize the sonic motion to the ultrasonic signal. Ultrasonic transducers had a PZT4 ceramic disk element with an active 6.4 mm diameter, a brass matching layer and an acrylic housing 9.5 mm dia. and 6.4 mm long. An applicator ring was molded with the same material as the Opal applicator tip in a comparable shape but with a 6.4 mm central hole to expose the ultrasonic module.

To synchronize the sonic and ultrasonic signal a custom Opal unit was developed. An encoder was embedded in this unit such that the position of the motor arm could be detected. When the armature was at rest the encoder indicated this as the center point. When the motor arm moved outward (and hence the applicator tip moving toward the skin) the encoder recorded this as positive movement and sent a trigger signal. This trigger signal was sent to a function generator and used to activate an ultrasonic pulse. Using the delay feature of the function generator, the location of the ultrasonic pulse with respect to the position of the motor arm (and hence the applicator tip) could be controlled.

Ultrasonic intensity levels were determined by first measuring the acoustic output (MPa, in water) with a hydrophone and then calculating the intensity (I_{SPTA}: spatial peak, temporal average intensity, I_{SPTA}: spatial peak, pulse average intensity, Mechanical Index, Acoustic Power, Average Acoustic Power) for the acoustic field. Note that these acoustic measures are made in water and have not been derated for other acoustic conditions.

The synchronization allowed for the adjustment of the ultrasonic parameter and triggered the waveform generator to create an ultrasonic pulse. The pulse was triggered synchronous to the sonic motion. Four positions of the ultrasonic pulse were tested: 1) “FCN”—centered about the point when the tip was closest to the skin, 2) “BCN”—centered about the point when the tip was furthest from the skin, 3) “FWD”—occurring as the tip is moving forward toward the skin and immediately before the tip reaches the point closest to the skin, and 4) “BCK”—occurring as the tip is moving away from the skin and immediately after the tip reaches the point closest to the skin. Note that the tip remains in contact with the skin surface at all times and moves inward and outward from the central position. For a non-pulsed, “continuous” ultrasonic condition the ultrasonic signal was active during the complete sonic cycle.

### 2. Conditions And Results

The results indicate an effect on absorption due to treatment condition. Note that “relative absorption” in the graphs is a measure of the absorption of light due to the presence of blue dye in the skin. In these studies the blue dye or fluorescent marker is used as a surrogate for an active ingredient. It is believed that conditions that favor absorption of blue dye or fluorescent marker into the skin also favor absorption of an active ingredient. Additionally for these tests the baseline condition is a sonic signal only device (Clarisonic Opal). It is anticipated that results obtained with other treatments are relative to the sonic only treatment are thus proportional or scalable as appropriate.

### A. Part 1

Purpose: Part 1 was a pilot experiment and investigated the ability of a device with combined sonic and ultrasonic vibrations to enhance absorption as compared to manual and sonic only treatments. Further two ultrasonic frequencies were compared and 4 different ultrasonic burst points within the sonic cycle (at this point the ultrasonic burst was not synchronized to a particular point within the sonic cycle, rather only the relative position within the cycle was varied).

### Test Variables:

TABLE 1
Treatment conditions - Part 1

| | | Ultrasonic | Ultrasonic duty | Ultrasonic | Phase between |
|---|---|---|---|---|---|
| | | Frequency | cycle/cycles | Intensity I_{STPA} | sonic and |
| Code | Treatment | (MHz) | per pulse | (mW/cm²) | ultrasonic pulse |
| P1-A | Manual, 30 s | — | — | — | — |
| P1-B | Sonic, 30 s | — | — | — | — |
| P1-C | Sonic + US, 30 s | 5.9 | 20%/9292 | 386 | 0 |
| P1-D | Sonic + US, 30 s | 5.9 | 20%/9292 | 386 | 90 |
| P1-E | Sonic + US, 30 s | 5.9 | 20%/9292 | 386 | 180 |
| P1-F | Sonic + US, 30 s | 5.9 | 20%/9292 | 386 | 270 |
| P1-G | Sonic + US, 30 s | 7.9 | 20%/12,504 | 384 | 0 |
| P1-H | Sonic + US, 30 s | 7.9 | 20%/12,504 | 384 | 90 |
| P1-I | Sonic + US, 30 s | 7.9 | 20%/12,504 | 384 | 180 |
| P1-J | Sonic + US, 30 s | 7.9 | 20%/12,504 | 384 | 270 |

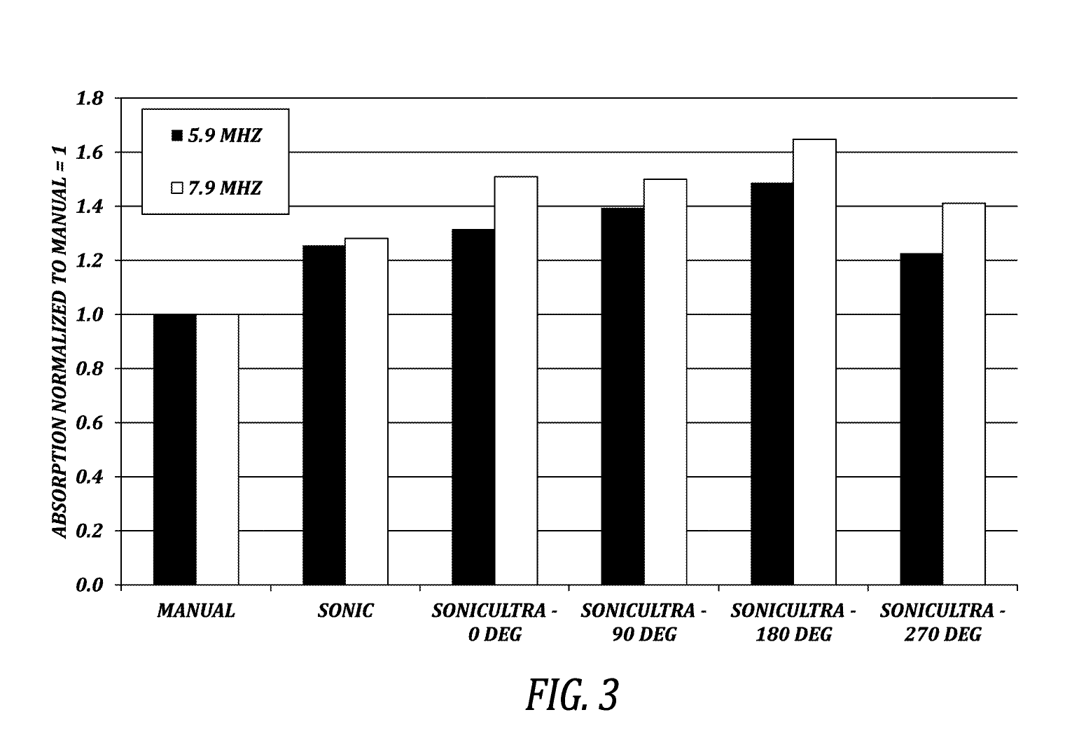

Results: The results are presented in FIG. 3. In this pilot experiment the results are normalized to the manual treatment for easier comparison.

B. Part 2
Purpose: Part 2 investigated the ability of a device with combined sonic and ultrasonic vibrations to enhance absorption as compared to manual, sonic only and ultrasonic only treatments.

### Test Variables:

TABLE 2A
Treatment conditions - Part 2

| | | Ultrasonic | Ultrasonic duty | Ultrasonic | Location of ultrasonic |
|---|---|---|---|---|---|
| | | Frequency | cycle/cycles | Intensity I_{STPA} | pulse within sonic |
| Code | Treatment | (MHz) | per pulse | (mW/cm²) | cycle |
| P2-F | Manual, 60 s | — | — | — | — |
| P2-G | Sonic, 60 s | — | — | — | — |
| P2-H | Ultrasonic, 60 s | 7.5 | 20%/11,811 | 693 | — |
| P2-I | Sonic + US-FCN, 60 s | 7.5 | 20%/11,811 | 693 | Centered forward |

TABLE 2B
Treatment condition ultrasonic intensity measures-Part 2

| | Peak | | Ultrasonic | Ultrasonic | | | Average |
|---|---|---|---|---|---|---|---|
| | Negative | Ultrasonic | Intensity | Intensity | | Acoustic | Acoustic |
| | Pressure | DC/cycles | I_{STPA} | I_{SPPA} | Mechanical | Power | Power |
| Code | (MPa) | per pulse* | (mW/cm²) | (W/cm²) | Index | (W) | (W) |
| P2-H, I | 0.32 | 20%/11,811 | 693 | 3.47 | 0.12 | 0.26 | 0.052 |

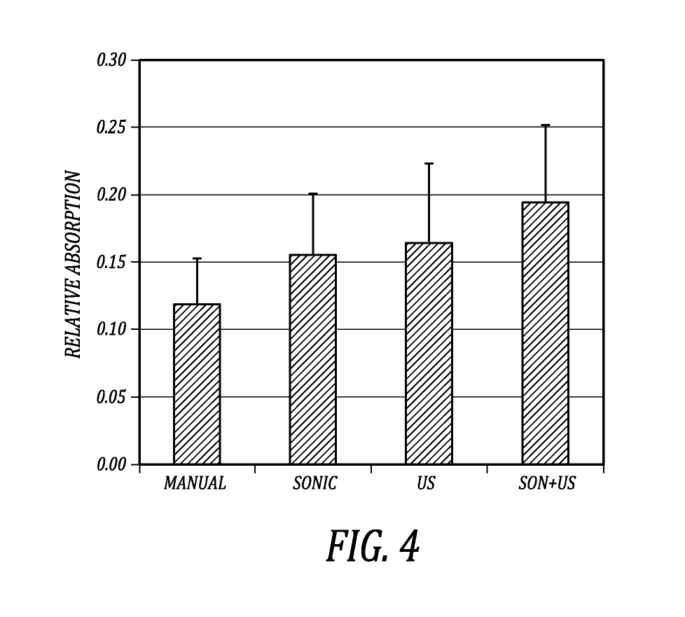

Results: The results are presented in FIG. 4.

### C. Part 3

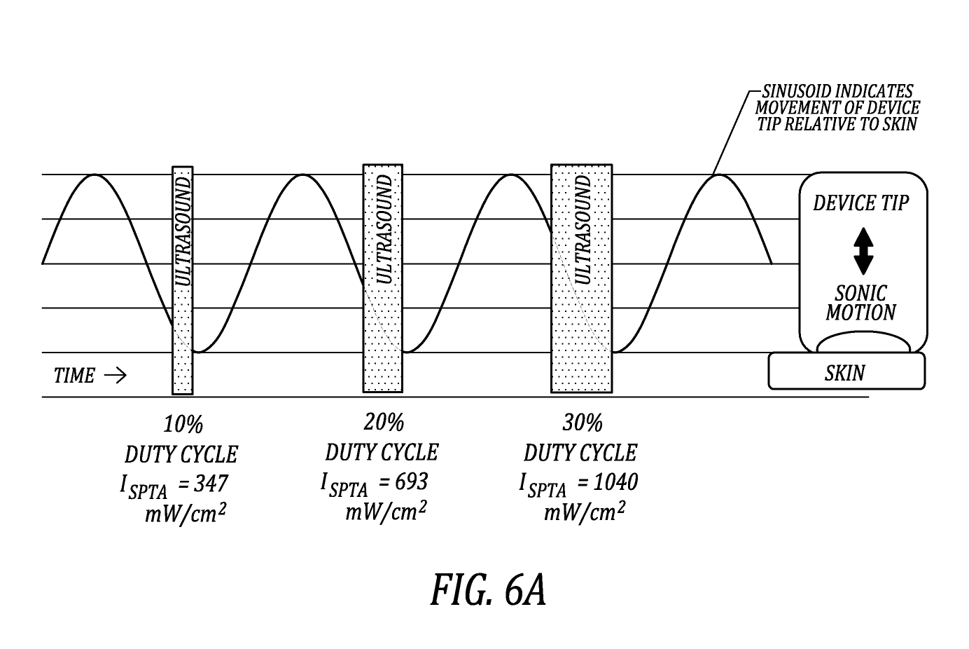

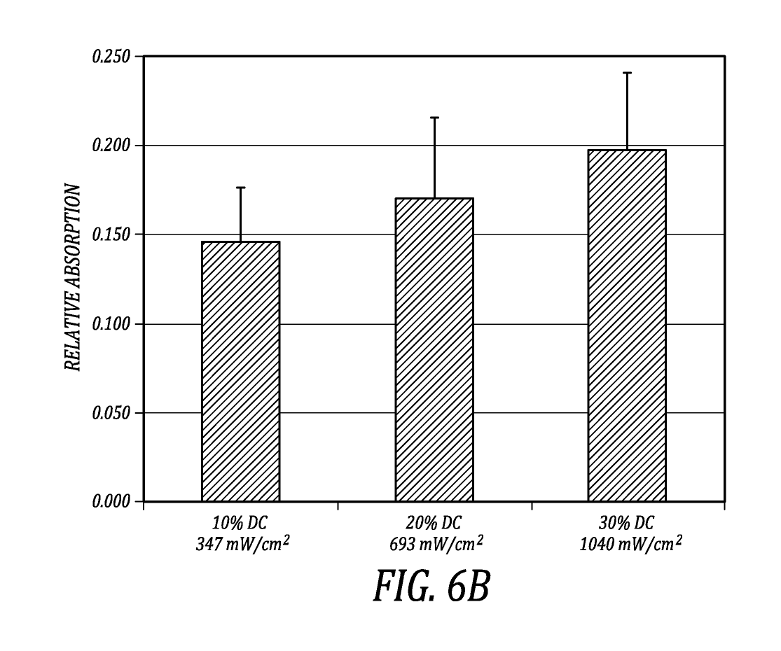

Purpose: Part 3 had two subparts: Part 3A investigated the synchronization between the ultrasonic burst and position of the sonic applicator tip. FIG. 5A graphically describes the burst positions with respect to the sonic cycle. Part 3B investigated the effect of the ultrasonic duty cycle (with a fixed acoustic pressure and variable acoustic intensity). FIG. 6A graphically describes the duty cycle differences.

### Test Variables:

TABLE 3A
Treatment conditions - Part 3

| | | Ultrasonic DC/ | Ultrasonic | |
|---|---|---|---|---|
| | | cycles per | Intensity I_{STPA} | Location of ultrasonic pulse |
| Code | Treatment (sonic + US) | pulse** | (mW/cm²) | within sonic cycle |
| P3-K | 10% DC-FWD, 60 s | 10%/5,906 | 347 | Moving forward to point |
| | | | | closest to skin |
| P3-L | 20% DC-FWD, 60 s | 20%/11,811 | 693 | Moving forward to point |
| | | | | closest to skin |
| P3-M | 20% DC-BCK, 60 s | 20%/11,811 | 693 | Moving backward from point |
| | | | | closest to skin |
| P3-N | 20% DC-FCN, 60 s | 20%/11,811 | 693 | Centered - closest to skin |
| P3-O | 20% DC-BCN, 60 s | 20%/11,811 | 693 | Centered - closest to skin |
| P3-P | 30% DC-FWD, 60 s | 30%/17,717 | 1040 | Centered - closest to skin |

**Ultrasonic frequency for all conditions: = 7.5 MHz

TABLE 3B
Treatment condition ultrasonic intensity measures-Part 3

| | Peak | | Ultrasonic | Ultrasonic | | | Average |
|---|---|---|---|---|---|---|---|
| | Negative | Ultrasonic | Intensity | Intensity | | Acoustic | Acoustic |
| | Pressure | DC/cycles | I_{STPA} | I_{SPPA} | Mechanical | Power | Power |
| Code | (MPa) | per pulse* | (mW/cm²) | (W/cm²) | Index | (W) | (W) |
| P3-K | 0.32 | 10%/5,906 | 347 | 3.47 | 0.12 | 0.26 | 0.026 |
| P3-L, M, | 0.32 | 20%/11,811 | 693 | 3.47 | 0.12 | 0.26 | 0.052 |
| N, O | | | | | | | |
| P3-P | 0.32 | 30%/17,717 | 1040 | 3.47 | 0.12 | 0.26 | 0.078 |

Results: The results are presented in FIG. 5B for synchronization variations and in FIG. 6B for the duty cycle variations.

### D. Part 4

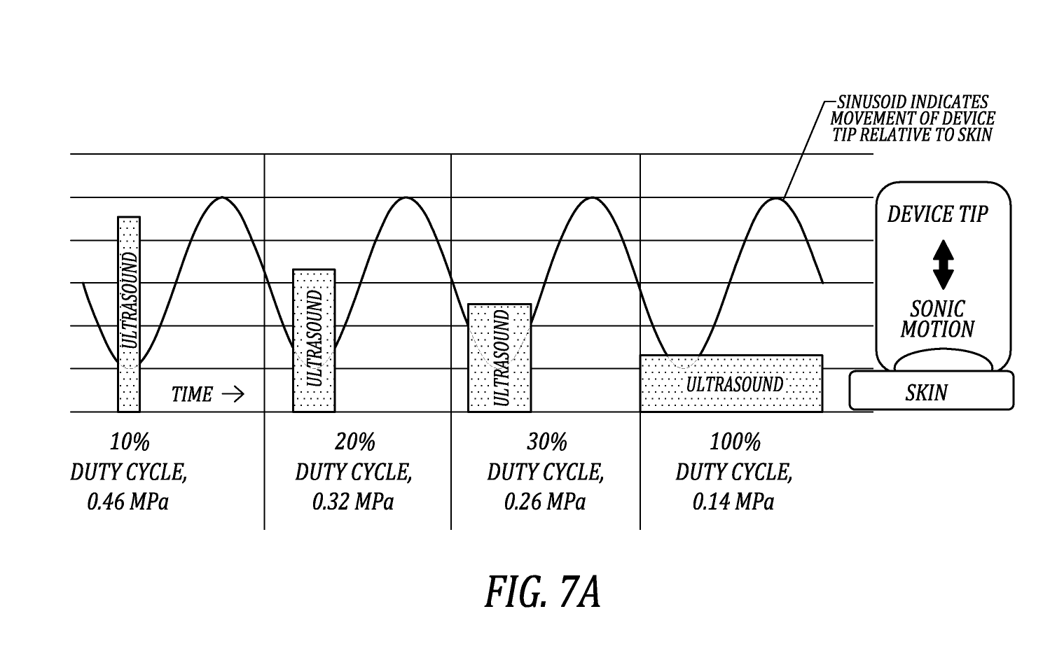

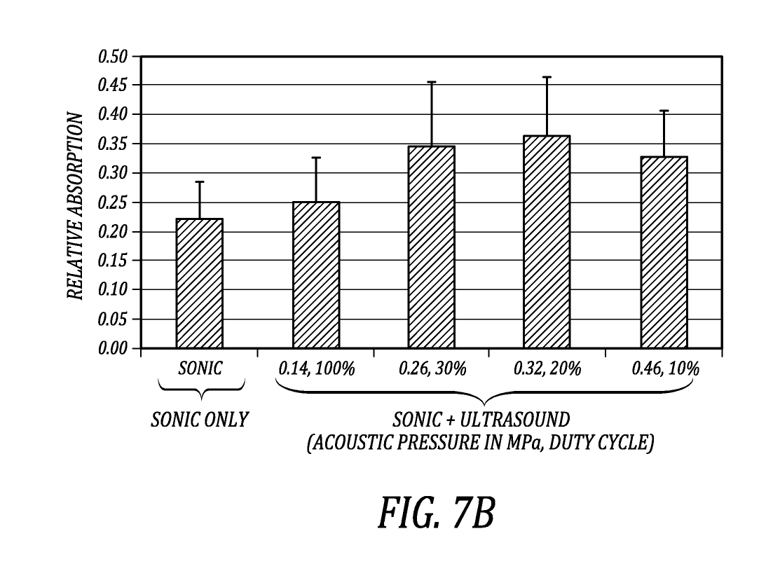

Purpose: Part 4 further investigated the ultrasonic duty cycle (with a variable acoustic pressure and fixed acoustic intensity) and used a sonic only comparative. FIG. 7A graphically describes the duty cycle and acoustic pressure variations.

### Test Variables:

TABLE 4A
Treatment conditions - Part 4

| | | Ultrasonic duty | Ultrasonic Peak | |
|---|---|---|---|---|
| | Treatment | cycle/cycles | Negative Pressure | Location of ultrasonic pulse |
| Code | (P4 Q-T sonic + US) | per pulse | (MPa)** | within sonic cycle |
| P4-Q | 0.46 MPa, 10% DC, 60 s | 10%/6,110 | 0.46 | Centered - closest to skin |
| PA-R | 0.32 MPa, 20% DC, 60 s | 20%/12,220 | 0.32 | Centered - closest to skin |
| P4-S | 0.26 MPa, 30% DC, 60 s | 30%/18,331 | 0.26 | Centered - closest to skin |
| P4-T | 0.14 MPa, 100% DC | 100%/ | 0.14 | Continuous |
| | (continuous US), 60 s | continuous | | |
| P4-U | Sonic, 60 s | — | — | — |

**Ultrasonic frequency for all conditions: = 7.7 MHz

TABLE 4B
Treatment condition ultrasonic intensity measures-Part 4

| | Peak | | Ultrasonic | Ultrasonic | | | Average |
|---|---|---|---|---|---|---|---|
| | Negative | Ultrasonic | Intensity | Intensity | | Acoustic | Acoustic |
| | Pressure | DC/cycles | I_{STPA} | I_{SPPA} | Mechanical | Power | Power |
| Code | (MPa) | per pulse* | (mW/cm²) | (W/cm²) | Index | (W) | (W) |
| P4-Q | 0.46 | 10%/6,110 | 693 | 6.91 | 0.17 | 0.52 | 0.05 |
| P4-R | 0.32 | 20%/12,220 | 693 | 3.47 | 0.12 | 0.26 | 0.05 |
| P4-S | 0.26 | 30%/18,331 | 693 | 2.32 | 0.09 | 0.17 | 0.05 |
| P4-T | 0.14 | 100%/continuous | 693 | 0.69 | 0.05 | 0.05 | 0.05 |

Results: The results are presented in FIG. 7B.

### E. Part 5

Purpose: Part 5 investigated the ability of sonic+ultrasonic to enhance absorption as compared to sonic only treatment. The methodology here employed a fluorescent dye in the applied formulation and tape stripping of the skin to assess absorption. Tape stripping removes one layer of stratum corneum cells per strip and allows quantification of absorption per cell layer. Absorption was assessed using the fluorescent properties of the marker.

### Test Variables:

TABLE 5A
Treatment conditions - Part 5

| | | Ultrasonic duty | Ultrasonic Peak | |
|---|---|---|---|---|
| | | cycle/cycles | Negative Pressure | Location of ultrasonic pulse |
| Code | Treatment | per pulse | (MPa)** | within sonic cycle |
| P5-V | Sonic, 60 s | — | — | — |
| P5-W | Sonic + US-FCN, 60 s | 20%/12,220 | 0.32 | Centered about point |
| | | | | closest to skin |

**Ultrasonic frequency for all conditions: = 7.7 MHz

TABLE 5B
Treatment condition ultrasonic intensity measures-Part 5

| | Peak | | Ultrasonic | Ultrasonic | | | Average |
|---|---|---|---|---|---|---|---|
| | Negative | Ultrasonic | Intensity | Intensity | | Acoustic | Acoustic |
| | Pressure | DC/cycles | I_{STPA} | I_{SPPA} | Mechanical | Power | Power |
| Code | (MPa) | per pulse* | (mW/cm²) | (W/cm²) | Index | (W) | (W) |
| P5-W | 0.32 | 20%/12,220 | 693 | 3.47 | 0.12 | 0.26 | 0.05 |

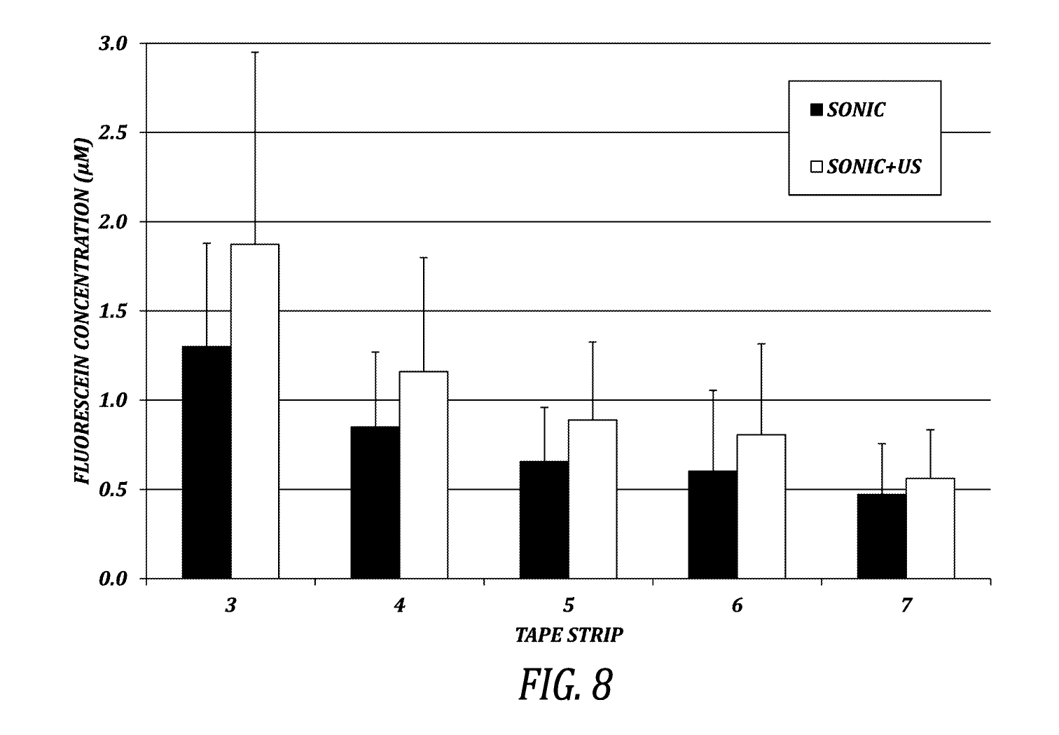

Results: The results are presented in FIG. 8 as concentration of absorbed fluorescein per tape strip as a result of treatment.

### 3. Conclusions

Both sonic treatment alone and ultrasonic treatment alone provided some improvement over manual treatment, as illustrated in FIG. 4. Combined sonic and ultrasonic treatment provided superior improvement over manual treatment than sonic only or ultrasonic only treatments. This synergy between the two is unique in that the sonic motion creates mechanical strain to open the pores, follicles, etc., while at the same time the ultrasonic motion acts to enhance absorption

Regarding the effect of the ultrasonic signal, when the acoustic pressure is kept constant, higher levels of ultrasound duty cycle (and thus higher acoustic intensity) yielded more relative absorption than lower duty cycle levels, as illustrated in FIG. 6B. By keeping the acoustic pressure constant, the ultrasonic driving action (pressure amplitude) is constant but the time of application (duty cycle) varies. It would be expected that with increased time of application the result would increase. Note that increasing the duty cycle increases I_{SPTA} (the temporal average intensity). There are recommended safety limits for derated I_{SPTA} of 720 mW/cm² (for diagnostic ultrasound, peripheral vessel). Note that the 30% duty cycle of FIG. 6B provides an I_{SPTA} of 1040 mW/cm² in water. Therefore simply increasing the duty cycle to get more absorption may not be desirable if the intensity reaches a safety limit.

Furthermore, when the acoustic intensity (I_{SPTA}) was held constant, bursts of ultrasound with higher acoustic pressure resulted in greater relative absorption than continuous ultrasound at a lower acoustic pressure, as illustrated in FIG. 7B.

If it is desired to remain below an I_{SPTA} of an established threshold, then the ultrasound must be delivered in a mode that maximizes impact while reducing intensity. Variables that may be adjusted include the duty cycle, the acoustic pressure and the point of synchronization within the sonic cycle. The results indicate that bursts of ultrasound with higher acoustic pressure yield more absorption than continuous ultrasound at a lower acoustic pressure. This suggests that partitioning and synchronization of the ultrasonic signal within the sonic cycle is important. Further, the trend toward higher absorption at 20% duty cycle indicates that the duration of ultrasound application is also important. It is likely that as the sonic action stretches the skin an optimal amount of time is necessary for the ultrasound to help drive the formulation into the openings within the skin. It appears that this optimal time may be greater than a 10% duty cycle but less than a 30% duty cycle.

Regarding the efficacy of treatment in relation to the position of the applicator tip relative to the skin, ultrasound bursting when the sonic tip was generally closer to the skin (FCN, FWD, and BCK) resulted in greater relative absorption than when the tip was further from the skin (BCN), as shown in FIG. 5B. This result indicates that location of the ultrasound burst within the sonic cycle influences the level of relative absorption. Ultrasound bursting when the sonic tip was generally closest to the skin resulted in the greatest amount of absorption for two reasons: (1) the skin is stretched the most under this condition and (2) the ultrasound applicator tip is closest to the skin resulting in the best acoustic coupling. The results suggest that synchronizing the two actions, sonic and ultrasonic, yields optimal results.

Ultrasound bursting when the sonic tip was centered about the point when the tip was closest to the skin (FCN) yielded greater relative absorption than other points studied.

Regarding ultrasound frequency, a frequency near 7.5 MHz generally yields greater relative absorption than an ultrasound frequency near 5 MHz. This result is shown in FIG. 3. It is possible that even higher frequencies may result in further enhancements in absorption, although ultrasound at higher frequencies may require more expensive generation methods.

A device with both sonic and ultrasonic motions was shown to increase absorption into the stratum corneum than a sonic only device as shown in FIG. 8. After treatment with the respective devices and tape stripping the skin, more fluorescent marker was observed within the stratum corneum (tape strips 3-7).

While illustrative embodiments have been illustrated and described, it will be appreciated that various changes can be made therein without departing from the spirit and scope of the invention.

The embodiments of the invention in which an exclusive property or privilege is claimed are defined as follows:

1. A dermal infusion device, comprising:
a first source of oscillatory motion, having an oscillation rate of less than 20 kHz; and
a second source of oscillatory motion, having an oscillation rate of 20 kHz or greater.

2. The device of claim 1, wherein the first source has an oscillation rate of less than 1 kHz.

3. The device of claim 1, wherein the first source is selected from the group consisting of a motor, a pneumatic device, and a piezoelectric device.

4. The device of claim 1, wherein the second source has an oscillation rate greater than 100 kHz.

5. The device of claim 1, wherein the second source is selected from the group consisting of a piezoelectric device and a magnetostrictive device.

6. The device of claim 1, wherein the second source creates bursts of oscillatory motion with a duty cycle between 5% and 50%.

7. The device of claim 1, wherein the second source has a spatial peak, temporal average intensity (I_{SPTA}) of 1 W/cm² or less.

8. The device of claim 1, wherein the first source has an amplitude of 0.1 inch or less.

9. The device of claim 1, wherein the first source and the second source are configured to simultaneously direct oscillatory motion to a location.

10. A method of simultaneously delivering a sonic motion and an ultrasonic motion to a location, comprising the steps of:
(a) directing the sonic motion at the location from a first source of oscillatory motion, having an oscillation rate of less than 20 kHz; and
(b) directing the ultrasonic motion at the location from a second source of oscillatory motion, having an oscillation rate of 20 kHz or greater.

11. The method of claim 10, wherein the location is a subject's skin.

12. The method of claim 10, wherein the sonic motion is produced by an oscillating contact member that oscillates between an extended position, closest to the location, and a retracted position, furthest from the location.

13. The method of claim 12, wherein the ultrasonic motion impinges upon the location and is delivered such that the ultrasonic motion is centered about the instance when the contact member is in the extended position.

14. The method of claim 12, wherein the ultrasonic motion impinges upon the location and is delivered when the contact member is moving from the retracted position to the extended position.

15. The method of claim 12, wherein the ultrasonic motion impinges upon the location and is delivered when the contact member is moving from the extended position to the retracted position.

16. The method of claim 10, further comprising applying a formulation to the location prior to the steps of directing the sonic motion and directing the ultrasonic motion.

17. The method of claim 16, wherein the formulation improves action between the location and the first source of oscillatory motion.

18. The method of claim 16, wherein the formulation improves action between the location and the second source of oscillatory motion.

19. The method of claim 16, wherein the formulation is a cosmetic composition.

20. The method of claim 16, wherein the formulation is a dermatological composition.
